(19) United States
(12) Patent Application Publication
(10) Pub. No.: US 2024/0376443 A1
(43) Pub. Date: Nov. 14, 2024

(54) NOVEL PROTEIN HAVING METHANE OXIDATION ACTIVITY

(71) Applicant: KOREA UNIVERSITY RESEARCH AND BUSINESS FOUNDATION, Seoul (KR)

(72) Inventors: Jee Won LEE, Seoul (KR); Yeon Hwa YU, Seoul (KR); Yoo Bin CHOI, Paju-si (KR)

(73) Assignee: KOREA UNIVERSITY RESEARCH AND BUSINESS FOUNDATION, Seoul (KR)

(21) Appl. No.: 18/288,490

(22) PCT Filed: Apr. 15, 2022
(86) PCT No.: PCT/KR2022/005480

§ 371 Date: Oct. 26, 2023

(30) Foreign Application Priority Data
Apr. 30, 2021 (KR) 10-2021-0056793

(51) Int. Cl. C12N 9/02; C07K 14/47; C12P 7/04

(52) U.S. Cl. CPC C12N 9/0071; C07K 14/47; C12N 9/0073; C12N 9/0083; C12P 7/04; C12Y 114/13025; C12Y 114/18003; C12Y 114/99039; C07K 2319/735

(57) ABSTRACT

The present invention relates to a protein including self-assembled ferritin monomers, in which a methane oxidation active domain, and an electron transfer domain including a flavin adenine dinucleotide (FAD)-binding domain are fused, and to: a protein which can oxidize methane by using NADH in vivo as a reducing agent, thus being capable of oxidizing methane to methanol without using a separate reducing agent; a microorganism expressing the same; a composition for producing methanol by using the same; and a method for producing methanol.

### TECHNICAL FIELD

The present invention relates to a novel protein having methane oxidation activity.

### BACKGROUND ART

Methane monooxygenase (MMO) derived from methanotrophs is a very useful biocatalyst that can catalyze the oxidation reaction of various hydrocarbons (C1-C8) including methane gas under mild conditions at room temperature and atmospheric pressure to produce high value-added products, therefore, global attention is focused on the development of a bioprocess using the above biocatalyst.

Further, as a similar enzyme, an ammonia oxidase (ammonia monoxygenase, AMO) derived from Nitrosomonas europaea is also a useful biocatlyst that has a similar mechanism to that of methane oxidase and can catalyze oxidation reactions for a wide range of hydrocarbons (C1-C10 chain/halogenated hydrocarbons, mono/polycyclic aromatic hydrocarbons). However, basic research on 3D structure, identification of active domains, reaction mechanism, substrate specificity, or the like is still very insufficient.

Currently, methanol production by a chemical process of methane gas is complicated in steps, and involves many problems in technical, environmental and economic aspects such as environmental pollution caused by the by-products (carbon dioxide, syngas, etc.), low reaction conversion rate, high energy consumption due to high temperature and high pressure reaction conditions or the like. Further, for methane gas, methanol production using a small-scale bio plant that can be easily connected to a local gas field is very advantageous in technical and economic aspects, due to problems such as economic degradation due to expensive transportation and storage costs, and occurrence of serious greenhouse effects when leaked.

As a result of efforts to develop bio-processes, metabolic engineering strain improvement of methane-oxidizing bacteria for the production of high value-added products other than methanol is being attempted, however, there are limitations in the use of genetic engineering tools and difficulties due to unculturable properties of the strain. Further, heterologous expression using industrial strains for mass production of methane oxidase entails a difficulty in expression of water-soluble protein and requires precise interaction of enzyme complexes, whereby no success case of industrial application is reported due to high technical difficulty.

### SUMMARY OF INVENTION

### Problems to be Solved by Invention

An object of the present invention is to provide a protein having excellent methane oxidation performance.

Another object of the present invention is to provide a microorganism expressing the protein.

In addition, another object of the present invention is to provide a composition for preparing methanol, including the protein or microorganism.

Further, another object of the present invention is to provide a method for production of methanol using the protein or microorganism.

### Means for Solving Problems

1. A protein including self-assembled ferritin monomers, in which a methane oxidation active domain and an electron transfer domain are fused.
2. The protein according to the above 1, wherein the methane oxidation active domain is pmoB1 (Particulate methane monooxygenase alpha subunit_domain 1), MMOH (Soluble methane monooxygenase hydroxylase), or amoB1 (Ammonia monooxygenase beta subunit_domain 1).
3. The protein according to the above 2, wherein the pmoB1 consists of an amino acid sequence of SEQ ID NO: 1, and the MMOH consists of an amino acid sequence of SEQ ID NO: 2 or 3.
4. The protein according to the above 2, wherein the amoB1 consists of an amino acid sequence of SEQ ID NO: 4.
5. The protein according to the above 1, wherein the electron transfer domain includes a flavin adenine dinucleotide (FAD)-binding domain.
6. The protein according to the above 1, wherein the electron transfer domain consists of an amino acid sequence of SEQ ID NO: 5 or 6.
7. The protein according to the above 1, wherein a ferritin monomer fused with the methane oxidation active domain and a ferritin monomer fused with the electron transfer domain including a FAD-binding domain are self-assembled.
8. The protein according to the above 1, wherein the ferritin monomer is further fused with MMOB (Methane monooxygenase regulatory protein B).
9. The protein according to the above 8, wherein the MMOB consists of an amino acid sequence of SEQ ID NO: 7 or 8.
10. The protein according to the above 8, wherein the methane oxidation active domain is MMOH (Soluble methane monooxygenase hydroxylase), and the MMOB is fused to a ferritin monomer fused with MMOH.
11. The protein according to the above 8, wherein the methane oxidation active domain is MMOH (Soluble methane monooxygenase hydroxylase), and a ferritin monomer to which the MMOH and the electron transfer domain are fused and a ferritin monomer to which MMOB is fused are self-assembled.
12. The protein according to the above 11, wherein the MMOB consists of an amino acid sequence of SEQ ID NO: 7 or 8.
13. The protein according to the above 1, wherein the ferritin monomer is a human ferritin heavy chain monomer.
14. The protein according to the above 1, wherein each domain is fused to any one selected from the group consisting of: inside α-helix of the ferritin monomer; between adjacent α-helices; N-terminus; C-terminus; A-B loop; B-C loop; C-D loop; D-E loop; between N-terminus and A helix; and between E helix and C-terminus.
15. A microorganism expressing the protein according to any one of the above 1 to 14.
16. The microorganism according to the above 15, wherein the microorganism is introduced with a vector which includes a gene encoding a ferritin monomer, a gene encoding a methane oxidation active domain, and a gene encoding an electron transfer domain which includes a flavin adenine dinucleotide (FAD)-binding domain.
17. The microorganism according to the above 16, wherein the gene encoding the methane oxidation active domain and the gene encoding the electron transfer domain which includes the FAD-binding domain are included in one vector or included in each of two vectors, respectively.
18. The microorganism according to the above 15, wherein the microorganism is E. coli.
19. The microorganism according to the above 15, wherein the microorganism further expresses formate dehydrogenase (FDH).
20. A composition for preparing methanol, including the protein according to any one of the above 1 to 14 or the microorganism according to any one of the above 15 to 19.
21. A method for producing methanol, including a step of reacting the composition according to the above 20 with methane gas.

### Advantageous Effects

The protein of the present invention has methane oxidation activity, and can use NADH in the living body as a reducing agent necessary for methane oxidation.

When preparing methanol in vivo using the protein of the present invention, it is not necessary to introduce a separate reducing agent.

### BRIEF DESCRIPTION OF DRAWINGS

FIGS. 1a-c are schematic diagrams of vectors used in each example.

FIG. 2 shows the expression rate and cytoplasmic solubility of recombinant proteins prepared in each example.

FIGS. 3a-b show results of confirming that the recombinant protein including cMMO, cAMO, and sMMO-mimics prepared in the examples forms a self-assembly.

FIGS. 4a to 6d show results of extended X-ray absorption fine structure (EXAFS), EPR and XANES analysis of recombinant proteins including cMMO, cAMO, and sMMO-m3 prepared in the examples.

FIG. 7 shows methane gas oxidation activity of the recombinant protein including cMMO, cAMO, and sMMO-mimics prepared in the examples.

FIG. 8 shows ¹³C-methane gas oxidation activity of cMMO, cAMO, and sMMO-m3 prepared in the examples.

FIG. 9 shows results of confirming the methane gas oxidation activity of a recombinant protein in which a methane oxidation active domain and an electron transfer domain including [2Fe-2S] and a FAD-binding domain are fused.

FIG. 10 shows results of confirming the methane gas oxidation activity of E. coli lysate expressing cAMO and sMMO-m3 proteins.

FIG. 11 shows results of confirming ¹³C-methane gas oxidation activity of E. coli lysate expressing cAMO and sMMO-m3 proteins.

### MODE FOR CARRYING OUT INVENTION

Hereinafter, the present invention will be described in detail.

The present invention relates to a protein including self-assembled ferritin monomer in which a methane oxidation active domain; and an electron transfer domain are fused.

The protein of the present invention may exhibit methane oxidation activity and electron transfer activity.

Methane may be oxidized to form methanol according to a reaction of Equation 1 below, and the protein of the present invention may include self-assembled ferritin monomers, in which a methane oxidation active domain; and an electron transfer domain are fused, wherein a methane oxidation reaction can be performed using NADH as a reducing agent, and in particular, NADH in the body can be utilized when the reaction proceeds in vivo. Accordingly, the use of a separate reducing agent is unnecessary.

CH₄+O₂+NAD(P)H+H⁺→CH₃OH+NAD(P)⁺+H₂O [Equation 1 ]

The methane oxidation active domain is a domain having an activity to oxidize methane, and any domain can be used as long as it is a domain including an active site in a methane oxidation enzyme or an active site of an enzyme having a methane oxidation ability by sequence or structural similarity, even if it is not derived from a methane oxidase.

In the present invention, as the methane oxidation active domain, for example, pmoB1 (particulate methane monooxygenase alpha subunit_domain 1), MMOH (soluble methane monooxygenase hydroxylase), amoB1 (ammonia monooxygenase beta subunit_domain 1), etc. may be used, but it is not limited thereto. The MMOH may be MMOHα. As a more particular example, the pmoB1 used herein may include an amino acid sequence of SEQ ID NO: 1. The MMOH used herein may include an amino acid sequence of SEQ ID NO: 2 or 3 Further, the amoB1 used herein may include an amino acid sequence of SEQ ID NO: 4.

The electron transfer domain may include, for example, a flavin adenine dinucleotide (FAD)-binding domain.

The FAD-binding domain may be derived from soluble MMO (methane monooxygenase) (sMMO), and specifically may be a FAD-binding domain (MMOR_{F}) of MMOR, which is one of its components.

The electron transfer domain may include a FAD-binding domain, which may consist of only the FAD-binding domain, or may further include an additional moiety in addition to the FAD-binding domain in the MMOR; may include at least a portion of the 2Fe-2S domain in addition to the FAD-binding domain; and may also include a FAD-binding domain and a 2Fe-2S domain. For example, the electron transfer domain used herein may include an amino acid sequence of SEQ ID NO: 5 or 6.

The protein of the present invention may include self-assembled ferritin monomers in which a methane oxidation active domain; and an electron transfer domain are fused, wherein each domain may be all fused to one ferritin monomer, may be fused to each ferritin monomer, or these domains may be mixed together.

That is, the protein of the present invention may include: a ferritin monomer fused with a methane oxidation active domain, and a ferritin monomer fused with an electron transfer domain, wherein these ferritin monomers are self-assembled; self-assembled ferritin monomers in which the above domains are all fused; or a ferritin monomer fused with all of the domains, and a ferritin monomer fused with one of the respective domains, wherein these ferritin monomers are self-assembled.

The ferritin monomer according to the present invention may include MMOB (Methane monooxygenase regulatory protein B) further fused therein.

The MMOB may be MMOB as a component of sMMO, for example, the MMOB may be MMOB including an amino acid sequence of SEQ ID NO: 7 or an amino acid sequence of SEQ ID NO: 8, but it is not limited thereto.

For example, the MMOB can be used together with MMOH (Soluble methane monooxygenase hydroxylase), which is a methane oxidation active domain. As a specific example in this case, MMOH, MMOB and electron transfer domains may be fused to one ferritin monomer, or split and fused to two ferritin monomers, separately. When split into two ferritin monomers, the MMOH and MMOB may be fused to one ferritin monomer, while the electron transfer domain may be fused to the other ferritin monomer. Alternatively, the MMOH and the electron transfer domain may be fused to one ferritin monomer, while the MMOB may be fused to the other ferritin monomer.

The protein of the present invention may include self-assembled ferritin monomers in which a methane oxidation active domain; and an electron transfer domain are fused. The ferritin monomer used herein may be ferritin derived from various organisms, and in the case of vertebrates, a heavy chain or light chain monomer may be used. For example, human ferritin heavy chains may be used.

The binding site of each domain is not limited as long as it can function in the self-assembled ferritin monomers of the protein, for example, the domain may be fused in any one site selected from the group consisting of: inside α-helix; between adjacent α-helices; N-terminus; C-terminus; A-B loop; B-C loop; C-D loop; D-E loop, between N-terminus and A helix; and between E helix and C-terminus, and, in an aspect of facilitating the function by being expressed externally from the protein, it is preferably fused at C-terminus.

In the protein of the present invention, a linker may be further included between the ferritin monomer and each domain.

As the linker, those known in the art may be used without limitation thereof, for example, S1 (G3SG3TG3SG3), S2 (GKLGGG), and the like may be used.

The protein of the present invention can be obtained from an organism after transforming, for example, a vector, which includes a gene encoding a ferritin monomer, a gene encoding a methane oxidation active domain, and a gene encoding an electron transfer domain including a flavin adenine dinucleotide (FAD)-binding domain, in the organism, but it is not limited thereto.

The protein of the present invention has a high expression rate in a soluble form in a microorganism, and thus has a high production yield during biosynthesis.

Further, the present invention relates to a microorganism expressing the protein.

The microorganism of the present invention may be introduced with a vector, which includes a gene encoding a ferritin monomer, a gene encoding a methane oxidation active domain, and a gene encoding an electron transfer domain, so as to express the protein.

In the protein of the present invention, each domain may be all fused to one ferritin monomer; may be fused to each ferritin monomer; or these domains may be mixed, so that the gene encoding the methane oxidation active domain and the gene encoding the electron transfer domain which includes the FAD-binding domain may be included in one vector or included in two vectors, respectively.

As the vector, an expression vector known in the art may be used, for example, BLUESCRIPT vector (Stratagene), T7 expression vector (Invitrogen), pET vector (Novagen), etc. may be included, but it is not limited thereto.

The vector may further include additional elements known in the art, such as a promoter for protein expression, a tag for isolation/purification, and a transform marker.

The type of microorganism is not limited as long as the vector can be introduced to express the protein, and for example, E. coli may be used.

Since the microorganism of the present invention expresses the protein, it can be utilized to produce methanol by oxidation of methane. When the microorganism of the present invention is used, it is not necessary to add a separate reducing agent for methanol production.

The microorganism of the present invention may further express formate dehydrogenase (FDH). The FDH may reduce intracellular NAD+ to NADH, thereby increasing the reducing ability through NADH reuse.

The microorganism of the present invention may be adapted to express FDH by introducing a vector containing a gene encoding FDH. The gene encoding FDH may be included in a vector including the gene encoding the ferritin monomer, otherwise, may be included in a separate vector.

Further, the present invention relates to a composition for producing methanol, which includes the above-described protein or the microorganisms.

Since the above-described protein has methane oxidation activity and electron transfer activity while the above-described microorganism expresses the protein, the composition of the present invention may oxidize methane to produce methanol.

Methane production may be performed by treating the composition with methane gas. When the protein is used, it should be treated with an additional reducing agent. Further, when the microorganism is used, the use of the reducing agent is unnecessary. The additional reducing agent may further include NADH.

In addition, the present invention relates to a method for production of methanol which includes reacting the above composition with methane gas.

Methanol may be produced by reacting the composition according to the present invention with methane gas to oxidize methane, which may be performed by injecting methane gas into the above-described composition and performing an enzymatic reaction.

Conditions for methanol production are not particularly limited and, for example, may be performed under conditions such as temperature and pH at which the above-described protein or microorganism exhibits appropriate activity.

### EXAMPLE

### 1. Preparation of Expression Vector for Protein Biosynthesis

According to the vector schematic view described in Table 1 below, chimeric MMO (pMMO(pmoB1)+sMMO(MMOR_{F})), chimeric AMO (AMO(amoB1)+sMMO(MMOR_{F})), sMMO-mimics (sMMO-m1 to sMMO-m5), cMMO(Full reductase, FR) (pMMO(pmoB1)+sMMO(MMOR)), cAMO(FR) (AMO(amoB1)+sMMO(MMOR)), and sMMO-m2(FR) were fabricated through PCR. All the fabricated plasmid expression vectors were purified on an agarose gel, followed by investigating the sequence through complete DNA sequencing.

The PCR products prepared as described above were sequentially inserted into pT7-7 and pET28a expression vectors to construct expression vectors capable of expressing each protein.

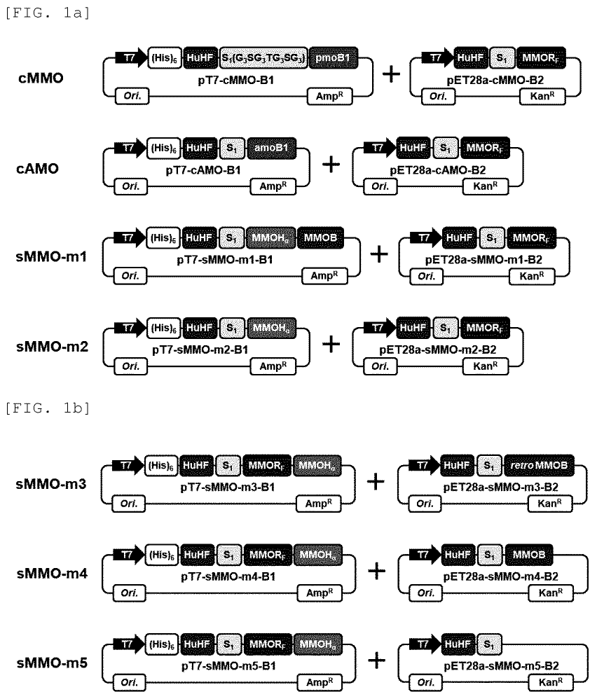

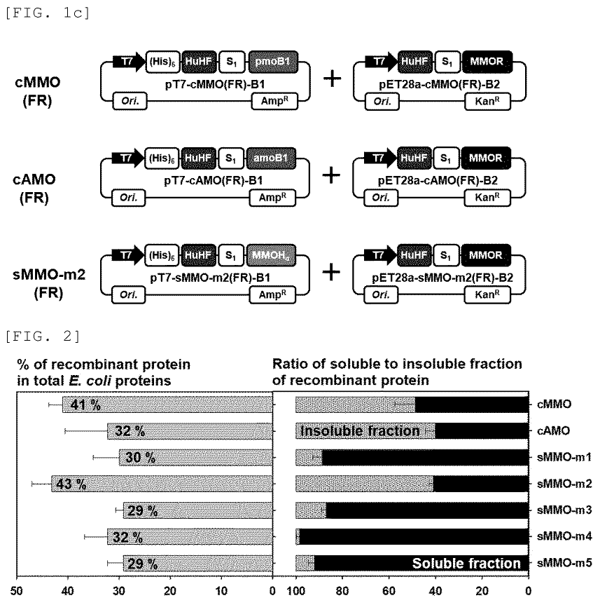

Vectors for expression of individual proteins were performed to pT7-cMMO-B1, pET28a-cMMO-B2, pT7-cAMO-B1, pET28a-cAMO-B2, pT7-sMMO-m1-B1, pET28a-sMMO-m1-B2, pT7-sMMO-m2-B1, pET28a-sMMO-m2-B2, pT7-sMMO-m3-B1, pET28a-sMMO-m3-B2, pT7-sMMO-m4-B1, pET28a-sMMO-m4-B2, pT7-sMMO-m5-B1, pET28a-sMMO-m5-B2, pT7-cMMO(FR)-B1, pET28a-cMMO(FR)-B2, pT7-cAMO(FR)-B1, pET28a-cAMO(FR)-B2, pT7-sMMO-m2(FR)-B1 and pET28a-sMMO-m2(FR)-B2, respectively (FIGS. 1a-c).

TABLE 1

| Recombinant | |
|---|---|
| protein | Expression vector |
| cMMO | 1. NH₂-NdeI-H₆-huHF-S₁(G₃SG₃TG₃SG₃)-BamHI- |
| | pmoB1(H33-T172)-HindIII-COOH |
| | 2. NH₂-NdeI-huHF-S₁-BamHI-MMOR_{F}(C99-A348)- |
| | HindIII-COOH |
| cAMO | 1. NH₂-NdeI-H₆-huHF-S₁-BamHI-amoB1(H38-L177)- |
| | HindIII-COOH2. NH₂-NdeI-huHF-S₁-BamHI-MMOR_{F}- |
| | HindIII-COOH |
| sMMO-m1 | 1. NH₂-NdeI-H₆-huHF-S₁-XhoI-MMOH_{α}(A64-L321)- |
| | BamHI-MMOB-HindIII-COOH2. NH₂-NdeI-huHF-S₁- |
| | BamHI-MMOR_{F}-HindIII-COOH |
| sMMO-m2 | 1. NH₂-NdeI-H₆-huHF-S₁-BamHI-MMOH_{α}(V93-K295)- |
| | HindIII-COOH2. NH₂-NdeI-huHF-S₁-BamHI-MMOR_{F}- |
| | HindIII-COOH |
| sMMO-m3 | 1. NH₂-NdeI-H₆-huHF-S₁-XhoI-MMOR_{F}- |
| | BamHI-MMOH_{α}(A64-L321) -HindIII-COOH2. NH₂- |
| | NdeI-huHF-S₁-BamHI-MMOB(retro)-HindIII-COOH |
| sMMO-m4 | 1. NH₂-NdeI-H₆-huHF-S₁-XhoI-MMOR_{F}- |
| | BamHI-MMOH_{α}(A64-L321)-HindIII-COOH |
| | 2. NH₂-NdeI-huHF-S₁-BamHI-MMOB-HindIII-COOH |
| sMMO-m5 | 1. NH₂-NdeI-H₆-huHF-S₁-XhoI-MMOR_{F}- |
| | BamHI-MMOH_{α}(A64-L321)-HindIII-COOH2. NH₂- |
| | NdeI-huHF-S₁-HindIII-COOH |
| cMMO(FR) | 1. NH₂-NdeI-H₆-huHF-S₁-BamHI-pmoB1-HindIII-COOH |
| | 2. NH₂-NdeI-huHF-S₁-BamHI-MMOR-HindIII-COOH |
| cAMO(FR) | 1. NH₂-NdeI-H₆-huHF-S₁-BamHI-amoB1-HindIII-COOH |
| | 2. NH₂-NdeI-huHF-S₁-BamHI-MMOR-HindIII-COOH |
| sMMO-m2 | 1. NH₂-NdeI-H₆-huHF-S₁-BamHI-MMOH_{α}(V93-K295)- |
| (FR) | HindIII-COOH |
| | 2. NH₂-NdeI-huHF-S₁-BamHI-MMOR-HindIII-COOH |

The amino acid sequences of each protein used are shown in Table 2 below.

TABLE 2

| | | SEQ |
|---|---|---|
| | | ID |
| Domain | Sequence | NO. |
| pmoB1 | HGEKSQAAFMRMRTIHWYDLSWSKEKVKINETVEIKGKFHVFEGWPETVDE | 1 |
| (33-172) | PDVAFLNVGMPGPVFIRKESYIGGQLVPRSVRLEIGKTYDFRVVLKARRPG | |
| | DWHVHTMMNVQGGGPIIGPGKWITVEGSMSEFRNPVTT | |
| MMOH_{α} | AKEYARMEAVKDERQFGSLQDALTRLNAGVRVHPKWNETMKVVSNFLEVGE | 2 |
| (64-321) | YNAIAATGMLWDSAQAAEQKNGYLAQVLDEIRHTHQCAYVNYYFAKNGQDP | |
| | AGHNDARRTRTIGPLWKGMKRVFSDGFISGDAVECSLNLQLVGEACFTNPL | |
| | IVAVTEWAAANGDEITPTVFLSIETDELRHMANGYQTVVSIANDPASAKYL | |
| | NTDLNNAFWTQQKYFTPVLGMLFEYGSKFKVEPWVKTWDRWVYEDWGGIWI | |
| | GRL | |
| MMOH_{α} | VRVHPKWNETMKVVSNFLEVGEYNAIAATGMLWDSAQAAEQKNGYLAQVLD | 3 |
| (93-295) | EIRHTHQCAYVNYYFAKNGQDPAGHNDARRTRTIGPLWKGMKRVFSDGFIS | |
| | GDAVECSLNLQLVGEACFTNPLIVAVTEWAAANGDEITPTVFLSIETDELR | |
| | HMANGYQTVVSIANDPASAKYLNTDLNNAFWTQQKYFTPVLGMLFEYGSK | |
| amoB1 | HGERSQEPFLRMRTVQWYDIKWGPEVTKVNENAKITGKFHLAEDWPRAAAQ | 4 |
| (38-177) | PDFSFFNVGSPSPVFVRLSTKINGHPWFISGPLQIGRDYEFEVNLRARIPG | |
| | RHHMHAMLNVKDAGPIAGPGAWMNITGSWDDFTNPLKL | |
| MMOR_{F} | CRISFGEVGSFEAEVVGLNWVSSNTVQFLLQKRPDECGNRGVKFEPGQFMD | 5 |
| (99-348) | LTIPGTDVSRSYSPANLPNPEGRLEFLIRVLPEGRFSDYLRNDARVGQVLS | |
| | VKGPLGVFGLKERGMAPRYFVAGGTGLAPVVSMVRQMQEWTAPNETRIYFG | |
| | VNTEPELFYIDELKSLERSMRNLTVKACVWHPSGDWEGEQGSPIDALREDL | |
| | ESSDANPDIYLCGPPGMIDAACELVRSRGIPGEQVFFEKFLPSGAA | |
| MMOR | MQRVHTITAVTEDGESLRFECRSDEDVITAALRQNIFLMSSCREGGCATCK | 6 |
| (FR) | ALCSEGDYDLKGCSVQALPPEEEEEGLVLLCRTYPKTDLEIELPYTHCRIS | |
| | FGEVGSFEAEVVGLNWVSSNTVQFLLQKRPDECGNRGVKFEPGQFMDLTIP | |
| | GTDVSRSYSPANLPNPEGRLEFLIRVLPEGRFSDYLRNDARVGQVLSVKGP | |
| | LGVFGLKERGMAPRYFVAGGTGLAPVVSMVRQMQEWTAPNETRIYFGVNTE | |
| | PELFYIDELKSLERSMRNLTVKACVWHPSGDWEGEQGSPIDALREDLESSD | |
| | ANPDIYLCGPPGMIDAACELVRSRGIPGEQVFFEKFLPSGAA | |
| MMOB | SVNSNAYDAGIMGLKGKDFADQFFADENQVVHESDTVVLVLKKSDEINTFI | 7 |
| | EEILLTDYKKNVNPTVNVEDRAGYWWIKANGKIEVDCDEISELLGRQFNVY | |
| | DFLVDVSSTIGRAYTLGNKFTITSELMGLDRKLEDYHA | |
| MMOB | AHYDELKRDLGMLESTITFKNGLTYARGITSSVDVLFDYVNFQRGLLESIE | 8 |
| (retro) | DCDVEIKGNAKIWWYGARDEVNVTPNVNKKYDTLLIEEIFTNIEDSKKLVL | |
| | VVTDSEHVVQNEDAFFQDAFDKGKLGMIGADYANSNVS | |

### 2. Biosynthesis and Purification of Recombinant Proteins

E. coli strain BL21(DE3) [F ⁻omp T hsdS_{B} (rB⁻ mB⁻)] was transformed with the expression vectors prepared above, respectively. For cMMO, cAMO, sMMO-m1 to m5, cMMO(FR), cAMO(FR) and sMMO-m2(FR), E. coli strain BL21 was simultaneously transformed with two expression vectors, and transformants resistant to ampicillin and kanamycin were selected. The transformed E. coli was cultured in a flask (250 mL Erlenmeyer flask, 37° C., 150 rpm) including 50 mL of Luria-Bertani (LB) medium (containing 100 mg L⁻¹ ampicillin and 100 mg L⁻¹ kanamycin, 0.4 mM CuSO₄ or FeSO₄).

When the medium turbidity (OD₆₀₀) reached about 0.6, Isopropyl-β-D-thiogalactopyanosid (IPTG) (1 mM) was added to induce gene expression. After 14 hours of incubation at 20° C., the cultured E. coli was centrifuged at 5,000 rpm for 5 minutes to recover the cell precipitate, which in turn was suspended in 5 mL of a lysis solution (50 mM NaH₂PO₄, 300 mM NaCl, 10 mM imidazole, pH 8.0) and crushed using an ultrasonic crusher (Branson Ultrasonics Corp., Danbury, CT, USA). After crushing, centrifugation was performed at 13,000 rpm for 10 minutes to separate the supernatant and insoluble aggregates. The separated supernatant was first subjected to Ni²⁺-NTA affinity chromatography using the binding of histidine and nickel fused to the recombinant protein, and then the recombinant protein was concentrated and buffer-exchanged to obtain purified recombinant protein. Details of each step are as follows.

### 1) Ni²⁺-NTA Affinity Chromatography

In order to purify the recombinant protein, E. coli cultured in the same manner as specified above was recovered, cell pellets thereof were resuspended in 5 mL of a lysis solution (50 mM NaH₂PO₄, 300 mM NaCl, 10 mM imidazole, pH 8.0), and an ultrasonic crusher was used to crush cells. The crushed cell solution was centrifuged at 13,000 rpm for 10 minutes to separate only the supernatant, and then each recombinant protein was separated using a Ni²⁺-NTA column (Quiagen, Hilden, Germany) (Wash Buffer: 50 mM NaH₂PO₄, 300 mM NaCl, 50 mM imidazole, pH 8.0/Elution Buffer: 50 mM NaH₂PO₄, 300 mM NaCl, 250 mM imidazole, pH 8.0).

### 2) Concentration and Buffer Exchange

2 mL of recombinant protein eluted through Ni²⁺-NTA affinity chromatography was placed in an ultracentrifugal filter (Amicon Ultra 100K, Millipore, Billerica, MA) and centrifuged at 5,000 rpm until 1 ml of solution remained on the column. Thereafter, the buffer was exchanged with Tris buffer (20 mM Tris-HCl, 250 mM NaCl, pH 8.0).

### 3. Analysis of Expression Rate and Cytoplasmic Solubility of Recombinant Proteins Including Prepared cMMO, cAMO, and sMMO-Mimics

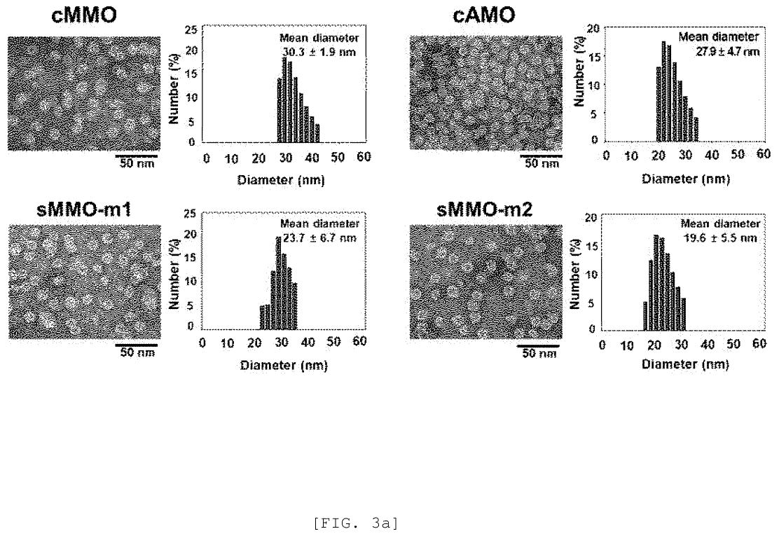

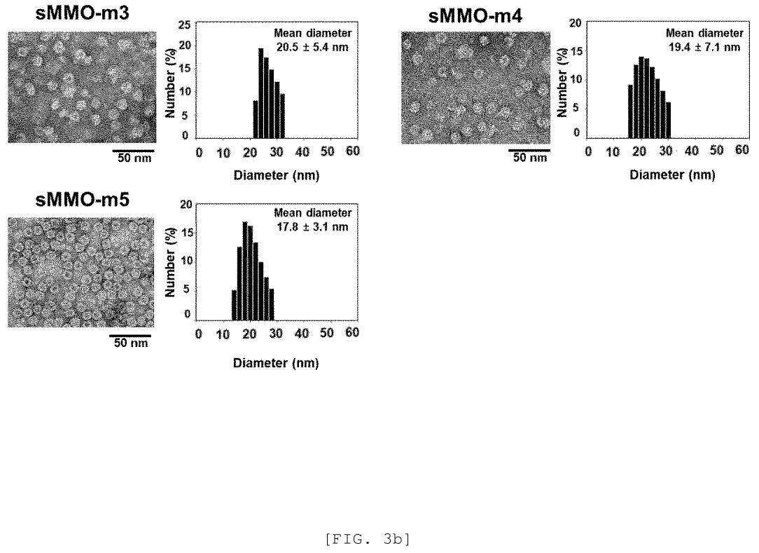

After the above process, the expression rate and cytoplasmic solubility of the purified recombinant protein were analyzed by SDS-PAGE. The supernatant (soluble fraction, sol), insoluble fraction (insol), and the purified recombinant protein obtained by centrifugation of the crushed cell solution of the recombinant protein were subjected to SDS-PAGE using 12% Tris-glycine precast gel (Invitrogen, California, U.S.A.). Then, the gel was stained with a Coomassie blue staining solution, and the expression rate and cytoplasmic solubility of each recombinant protein were analyzed using a densitometer (GS-800 Calibrated Densitometer, Bio-Rad, California, U.S.A.) for the stained protein band (FIG. 2).

### 4. Structural Analysis of Recombinant Proteins Including Prepared cMMO, cAMO, and sMMO-Mimics

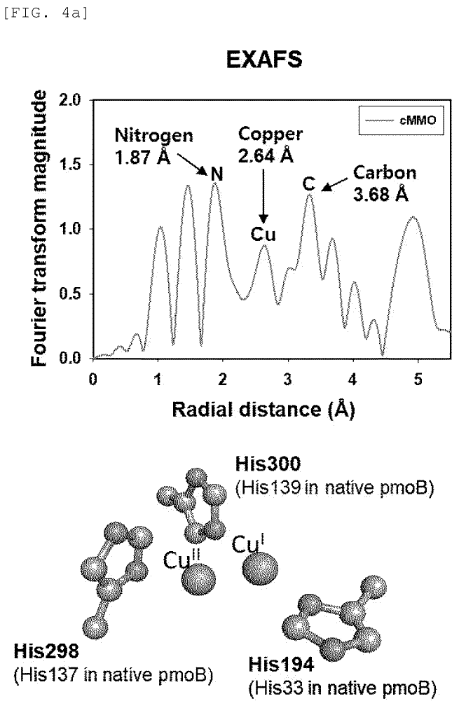

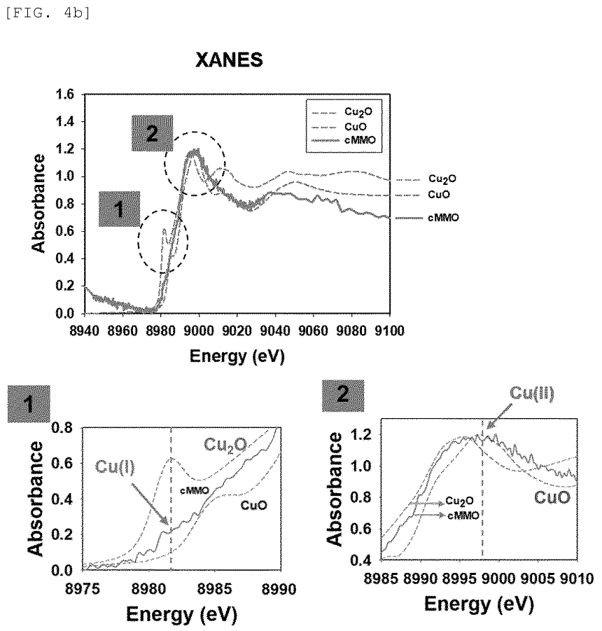

After the above process, transmission electron microscopy (TEM) imaging was performed to analyze the structure of the purified recombinant protein. To obtain stained images of proteins, electron microscope grids containing naturally dried samples were incubated with 2% (w/v) aqueous uranyl acetate solution for 1 hour at room temperature. The protein image was observed using a Tecnai 20 (FEI, Hillsboro, Oregon, U.S.A.) electron microscope operating at 200 kV, and as a result of observation, it was confirmed that a spherical self-assembly was formed. Additionally, through dynamic light scattering (DLS) analysis, it was confirmed that self-assemblies are formed with sizes of 30.3±1.9 nm for cMMO, 27.9±4.7 nm for cAMO, 23.7±6.7 nm for sMMO-m1, 19.6±5.5 nm for sMMO-m2, and sMMO-m3 is 20.5±5.4 nm for sMMO-m3, 19.4±7.1 nm for sMMO-m4, and 17.8±3.1 nm for sMMO-m5, respectively (FIGS. 3a-b).

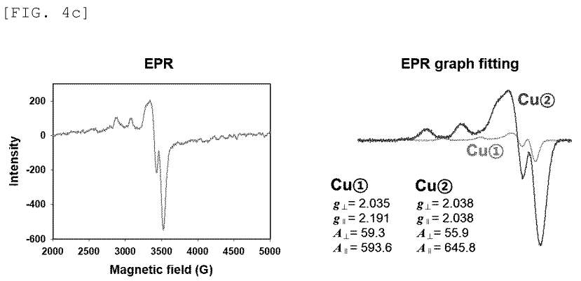

X-ray absorption spectroscopy (XAS) and electron paramagnetic resonance (EPR) spectra analysis were performed to analyze the structure of the recombinant protein including the prepared cMMO, cAMO and sMMO-m3. For X-ray absorption near-edge structure (XANES), extended X-ray absorption fine structure (EXAFS) and EPR analysis of proteins, the solvent-exchanged samples with Tris buffer were pre-frozen at −80° C. for 3 hours, and the pre-frozen samples were lyophilized at −110° C. using a lyophilizer (FDU-2100, DRC-1000, EYELA). XAS analysis was performed for measurement by XAFS beam line (BL11S2) of Aichi Synchrontron Radiation Center (Aichi), and EPR spectrometer (JES-FA200). As a result of cMMO EXAFS analysis, distance information between copper ions and surrounding ligands present in the active site was confirmed. Further, as a result of XANES analysis, it was confirmed that mono- and bivalent copper ions (Cu(I), Cu(II)) were mixed. Additionally, through EPR analysis, it was confirmed that the bivalent copper ions existed in a valence-scrambled state (FIGS. 4a-c).

As a result of cAMO EXAFS analysis, information on a distance between copper ions and surrounding ligands present in the active site was confirmed, and in the case of a sample subjected to the methane oxidation reaction, it was confirmed that the ligand distance was changed compared to a non-reacted sample, and one peak (up to 2.2 Å) was additionally observed. As a result of XANES analysis, it was confirmed that mono- or bivalent copper ions (Cu(I), Cu(II)) were mixed.

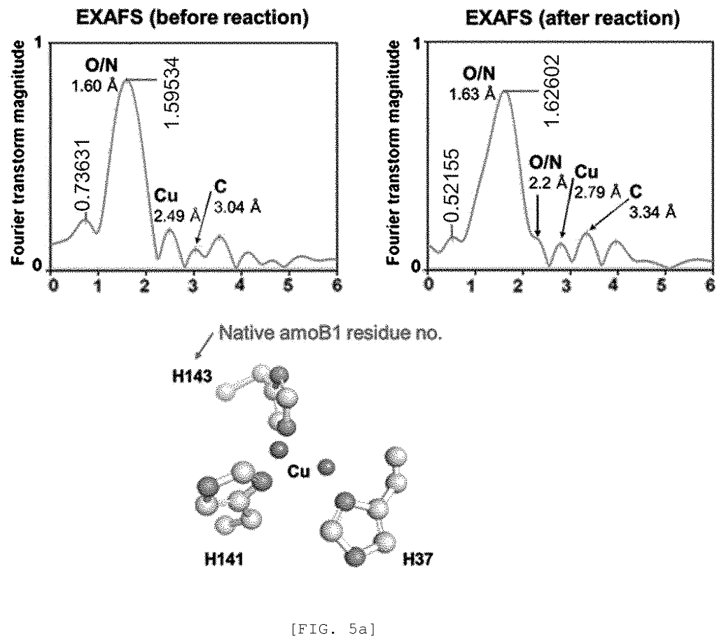

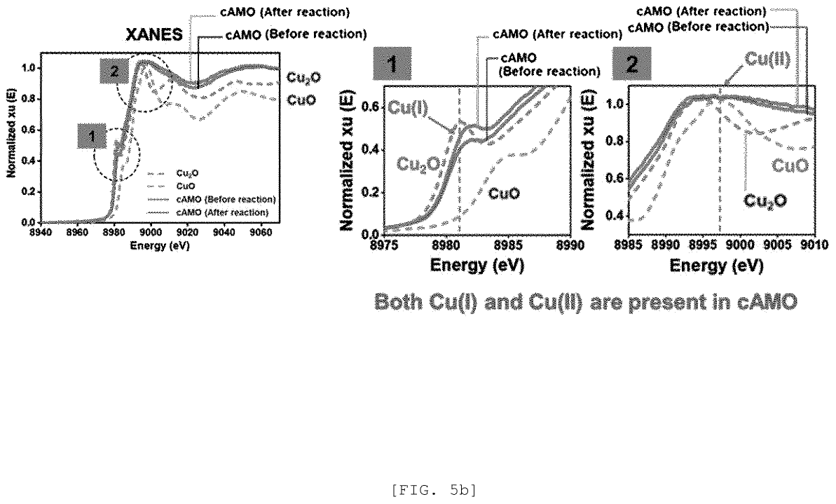

Additionally, through EPR analysis, it was confirmed that the bivalent copper ions existed in a valence-scrambled state (FIGS. 5a-c).

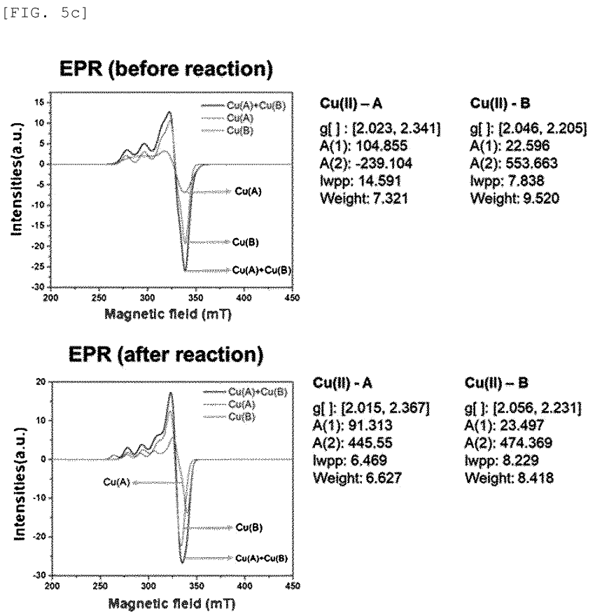

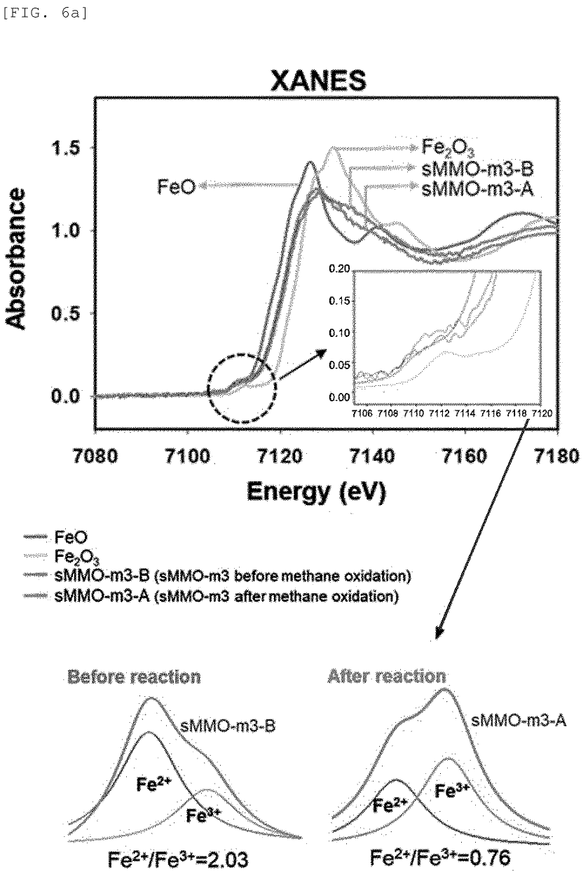

As a result of the sMMO-m3 EXAFS analysis, information on the distance between the iron ion and the surrounding ligand present at the active site was confirmed. Further, as a result of XANES and EPR analysis, in the case of a sample including bivalent and trivalent iron ions (Fe(II), Fe(III)) mixed therein and subjected to the methane oxidation reaction, it was confirmed that, compared to the non-reacted sample, the ratio of Fe(III)/Fe(II) was increased. Additionally, the presence of trivalent iron ions was confirmed through EPR analysis (FIGS. 6a-d).

### 5. Verification of Methane Gas Oxidation Activity of Recombinant Proteins Including Prepared cMMO, cAMO, and sMMO-Mimics

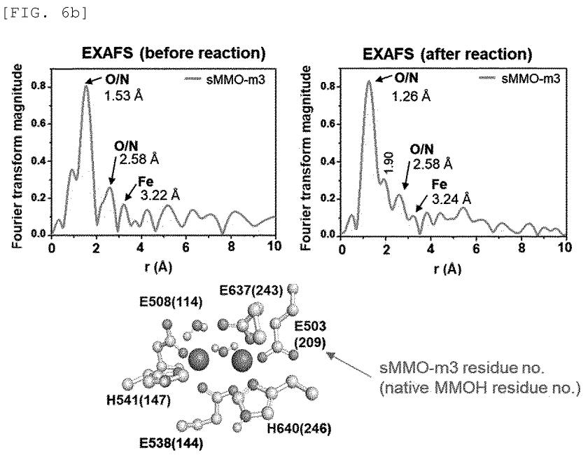

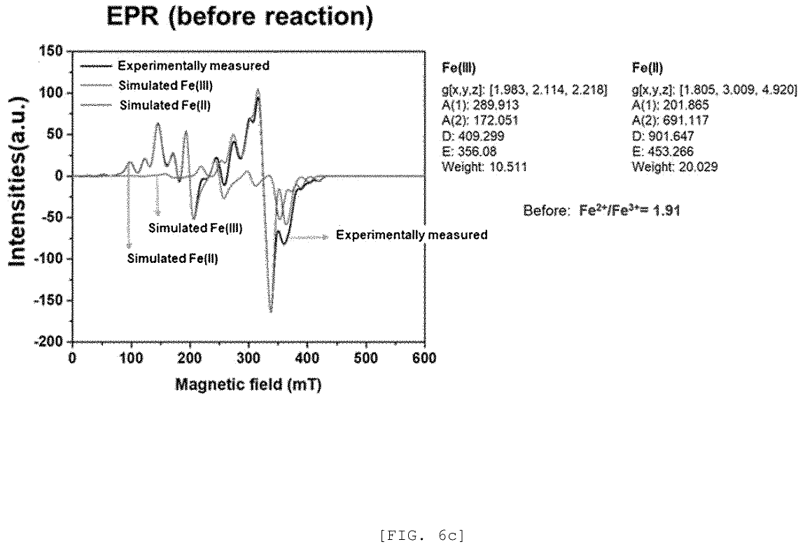

To verify the methane gas oxidation activity of the purified recombinant protein, 1 mL of a recombinant protein solution containing a reducing agent NADH (0.2 to 0.3 mM) was injected to a 20 mL septa-sealed vial (catalogue no. 5182-0837, Agilent). For the methane oxidation reaction through the recombinant protein, 19 mL of air in a headspace was removed through a syringe and 15 mL of methane gas and 4 mL of air were injected. Then, the vial was immediately subjected to the enzymatic reaction in an incubator at 30° C. for maximum 24 hours. Further, an amount of methanol as an oxidation product generated by the enzymatic reaction was measured through gas chromatography (7890B GC, Agilent) to calculate the cumulative production (FIG. 7).

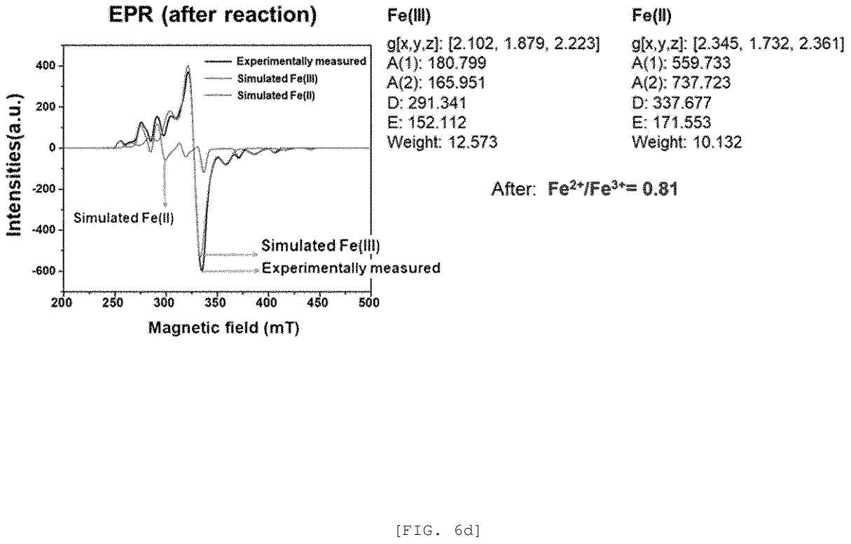

To verify the ¹³C-methane gas oxidation activity of cMMO, cAMO, and sMMO-m3, the enzymatic reaction was performed in the same manner as the methane oxidation reaction specified above, except that the methane gas was replaced with ¹³C-methane gas. For nuclear magnetic resonance (NMR) analysis, 5 vials after completing the reaction were heated at 80° C. for 15 minutes, followed by directly injecting 19 ml of headspace gas into 600 μl of sufficiently cooled ethanol using a syringe. Thereafter, 60 μl of ethanol-d₆ was added, transferred to an NMR tube (NORS55007, Sigma Aldrich), and ¹³C-methanol generated by the cMMO, cAMO, sMMO-m3 enzymatic reaction (which is the generated oxidation product) was confirmed by NMR analysis (FIG. 8).

### 6. Verification of Methane Gas Oxidation Activity of a Recombinant Protein in Which a Methane Oxidation Active Domain and an Electron Transfer Domain Including [2Fe-2S] and a FAD-Binding Domain are Fused

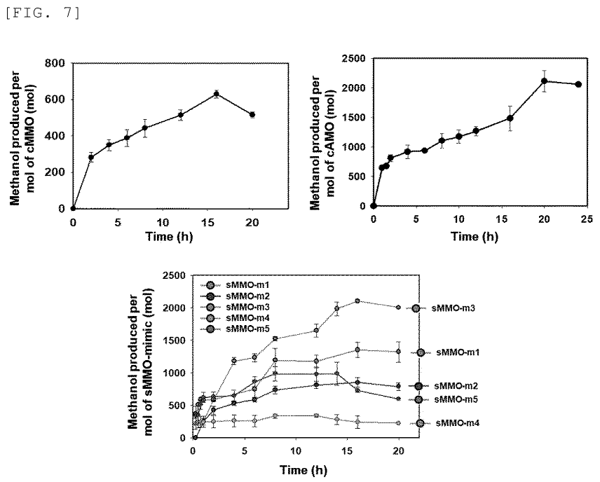

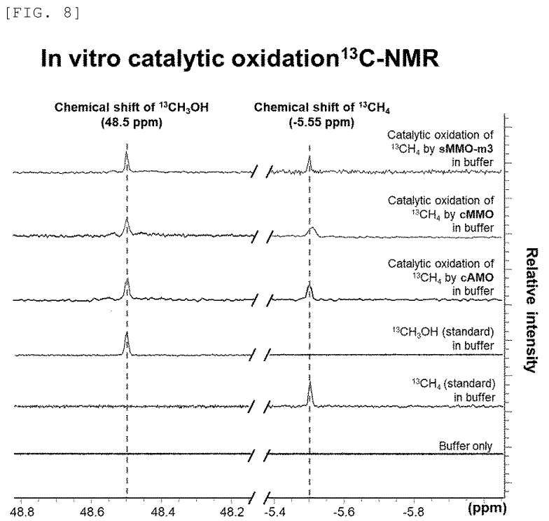

cMMO(FR), cAMO(FR) and sMMO-m2(FR) recombinant proteins, in which the electron transfer domain is substituted with an electron transfer domain including [2Fe-2S] and FAD-binding domains for cMMO, cAMO, and sMMO-m2, were prepared according to the method clarified above, and then, an amount of methanol production was determined through gas chromatography after 16 hours of methane gas oxidation reaction (cMMO(FR): ND, cMMO: 629.28(±21.23) mol methanol/mol enzyme, cAMO(FR): 427.03(±50.2) mol methanol/mol enzyme, cAMO: 1355.16(±213.06) mol methanol/mol enzyme, sMMO-m2(FR): 570.60(±50.96) mol methanol/mol enzyme, sMMO-m2: 886.62(±46.86) mol methanol/mol enzyme). Through comparison of methanol production, it was confirmed that, rather than the recombinant protein in which the electron transfer domain (MMOR(FR)) including both of [2Fe-2S] and the FAD-binding domain are fused, the recombinant protein in which the electron transfer domain (MMOR_{F}) containing only the FAD-binding domain is fused showed higher methane oxidation activity (FIG. 9).

### 7. Verification of Methane Gas Oxidation Activity of E. coli Lysate Expressing Recombinant Protein

To verify methane gas oxidation activity using E. coli expressing cMMO, cAMO sMMO-m3 proteins, the strain BL21(DE3) [ΔyrfE ΔyjaD::pncB] (Eng. Life Sci., 7, 343-353, 2007), into which the pncB gene having genetic information of the corresponding enzyme was introduced so that the enzyme synthesizing the NAD(H) precursor in E. coli cells (that is, nicotinic acid phosphoribosyl transferase) can be expressed, was used. Further, in order to increase the reducing ability through NADH reuse by reducing intracellular NAD⁺to NADH, Pseudomonas sp. (strain 101)-derived FDH (formate dehydrogenase) (1) Journal of solid-phase biochemistry, 5, 19-33, 1980, 2) Cell, 179(6), 1255-1263, 2019) and pETDuet-1 vector were used thus to construct an expression vector capable of being co-expressed with cMMO, cAMO and sMMO-m3 proteins (Table 3).

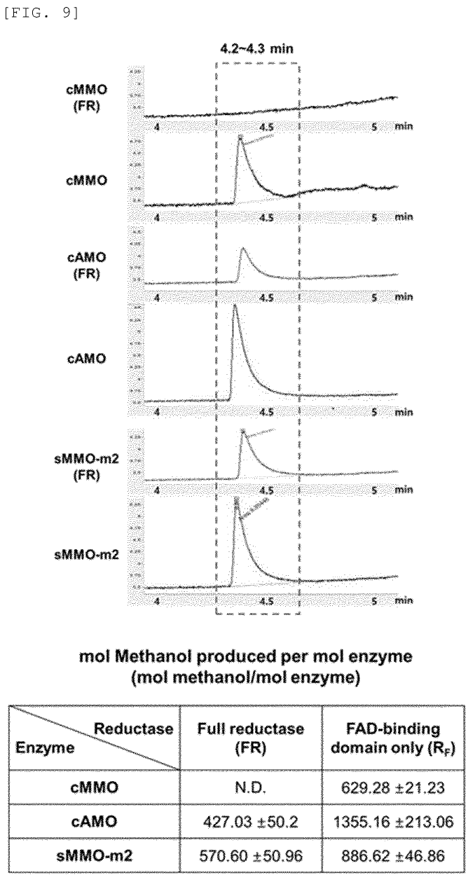

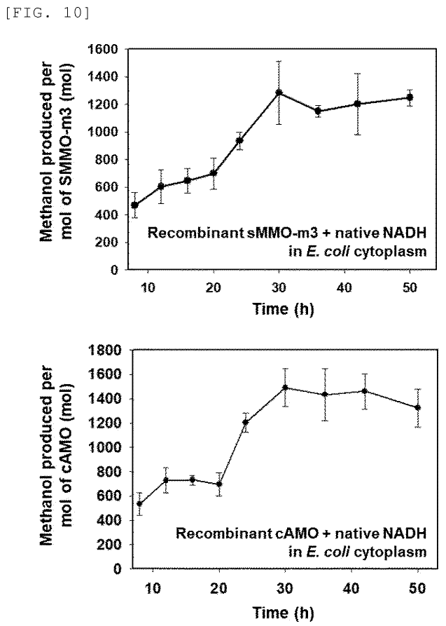

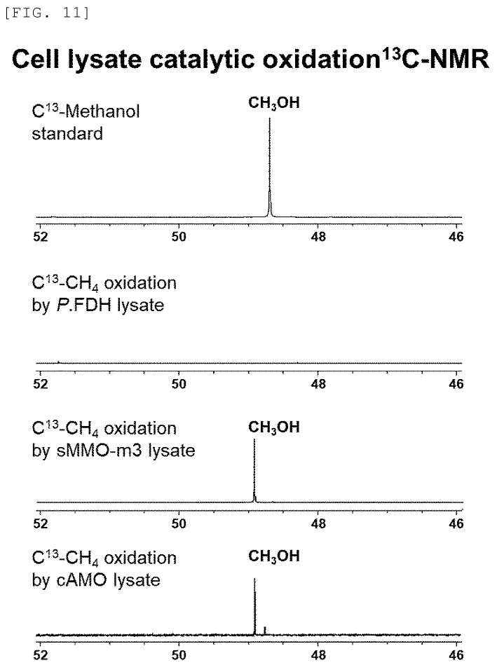

This strain (BL21(DE3) [ΔyrfEΔyjaD::pncB]) was transformed with vectors for expression of cMMO, cAMO and sMMO-m3 proteins, respectively, followed by performing protein expression through the culture method specified above. Further, during addition of 1 mM IPTG, 50 mM sodium formate was added to maintain the reducing ability in the strain, followed by culturing at 20° C. for 14 hours. The cultured E. coli was centrifuged at 4,500 rpm for 10 minutes to recover the cell sediment, and then resuspended in a lysis solution purged with methane gas, followed by crushing the cells using an ultrasonic crusher. The crushed cell lysate was suspended so that the E. coli turbidity (OD₆₀₀) before crushing becomes 30, and then was dispensed into GC vials by 3 mL each. For a methane oxidation reaction through E. coli lysate expressing recombinant protein, at first, air from 19 mL of vial headspace was removed by a syringe, and 10 mL of methane gas and 9 mL of air were injected. Then, the vial was immediately subjected to a lysate reaction in an incubator at 30° C. for up to 52 hours. The vial was heated at 80° C. for 20 minutes after completing the reaction so that the generated methanol was vaporized. Then, an amount of methanol as an oxidation product generated by lysate was measured through gas chromatography to calculate the cumulative production. (Maximum methanol production of sMMO-m3 expression lysate: 1280 MeOH (mol)/enzyme (mol), cAMO expression maximum methanol production: 1460 MeOH (mol)/enzyme (mol)) (FIG. 10).

TABLE 3

| Recombinant | |
|---|---|
| protein | Expression vector |
| cAMO | 1-1. NH₂-NcoI-H₆-huHF-S₁-BamHI-amoB1(H38-L177)- |
| (w/P.FDH) | HindIII-COOH |
| | 1-2. NH₂-NdeI-P.FDH-XhoI-COOH |
| | 2. NH₂₋NdeI-huHF-S₁-BamHI-MMOR_{F}-HindIII-COOH |
| sMMO-m3 | 1-1. NH₂₋ NcoI-H₆-huHF-S₁-XhoI-MMOR_{F}-BamHI- |
| (w/P.FDH) | MMOH_{α}(A64-L321)-HindIII-COOH |
| | 1-2. NH₂-NdeI-P.FDH-XhoI-COOH |
| | 2. NH₂-NdeI-huHF-S₁-BamHI-MMOB(retro)-HindIII- |
| | COOH |

1. A protein comprising self-assembled ferritin monomers, in which a methane oxidation active domain and an electron transfer domain are fused.

2. The protein according to claim 1, wherein the methane oxidation active domain is pmoB1 (Particulate methane monooxygenase alpha subunit_domain 1), MMOH (Soluble methane monooxygenase hydroxylase), or amoB1 (Ammonia monooxygenase beta subunit_domain 1).

3. The protein according to claim 2, wherein the pmoB1 consists of an amino acid sequence of SEQ ID NO: 1, and the MMOH consists of an amino acid sequence of SEQ ID NO: 2 or 3.

4. The protein according to claim 2, wherein the amoB1 consists of an amino acid sequence of SEQ ID NO: 4.

5. The protein according to claim 1, wherein the electron transfer domain includes a flavin adenine dinucleotide (FAD)-binding domain.

6. The protein according to claim 1, wherein the electron transfer domain consists of an amino acid sequence of SEQ ID NO: 5 or 6.

7. The protein according to claim 1, wherein a ferritin monomer fused with the methane oxidation active domain and a ferritin monomer fused with the electron transfer domain including a FAD-binding domain are self-assembled.

8. The protein according to claim 1, wherein the ferritin monomer is further fused with MMOB (Methane monooxygenase regulatory protein B).

9. The protein according to claim 8, wherein the MMOB consists of an amino acid sequence of SEQ ID NO: 7 or 8.

10. The protein according to claim 8, wherein the methane oxidation active domain is MMOH (Soluble methane monooxygenase hydroxylase), and the MMOB is fused to a ferritin monomer fused with MMOH.

11. The protein according to claim 8, wherein the methane oxidation active domain is MMOH (Soluble methane monooxygenase hydroxylase), and a ferritin monomer to which the MMOH and the electron transfer domain are fused and a ferritin monomer to which MMOB is fused are self-assembled.

12. The protein according to claim 11, wherein the MMOB consists of an amino acid sequence of SEQ ID NO: 7 or 8.

13. The protein according to claim 1, wherein the ferritin monomer is a human ferritin heavy chain monomer.

14. The protein according to claim 1, wherein each domain is fused to any one selected from the group consisting of: inside α-helix of the ferritin monomer; between adjacent α-helices; N-terminus; C-terminus; A-B loop; B-C loop; C-D loop; D-E loop; between N-terminus and A helix; and between E helix and C-terminus.

15. A microorganism expressing the protein according to claim 1.

16. The microorganism according to claim 15, wherein the microorganism is introduced with a vector which includes a gene encoding a ferritin monomer, a gene encoding a methane oxidation active domain, and a gene encoding an electron transfer domain which includes a flavin adenine dinucleotide (FAD)-binding domain.

17. The microorganism according to claim 16, wherein the gene encoding the methane oxidation active domain and the gene encoding the electron transfer domain which includes the FAD-binding domain are included in one vector or included in each of two vectors, respectively.

18. The microorganism according to claim 15, wherein the microorganism is E. coli.

19. The microorganism according to claim 15, wherein the microorganism further expresses formate dehydrogenase (FDH).

20. A composition for preparing methanol, comprising the protein according to claim 1.

21. A method for producing methanol, comprising a step of reacting the composition according to claim 20 with methane gas.

22. A composition for preparing methanol, comprising the microorganism according to claim 15.


SEQUENCE LISTING (8 sequences)

SEQ ID NO: 1 - protein, 140 aa, Methylococcus capsulatus
HGEKSQAAFMRMRTIHWYDLSWSKEKVKINETVEIKGKFHVFEGWPETVDEPDVAFLNVGMPGPVFIRKESYIGGQLVPRSVRLEIGKTYDFRVVLKARRPGDWHVHTMMNVQGGGPIIGPGKWITVEGSMSEFRNPVTT

SEQ ID NO: 2 - protein, 258 aa, Unknown
  methanotrophs
AKEYARMEAVKDERQFGSLQDALTRLNAGVRVHPKWNETMKVVSNFLEVGEYNAIAATGMLWDSAQAAEQKNGYLAQVLDEIRHTHQCAYVNYYFAKNGQDPAGHNDARRTRTIGPLWKGMKRVFSDGFISGDAVECSLNLQLVGEACFTNPLIVAVTEWAAANGDEITPTVFLSIETDELRHMANGYQTVVSIANDPASAKYLNTDLNNAFWTQQKYFTPVLGMLFEYGSKFKVEPWVKTWDRWVYEDWGGIWIGRL

SEQ ID NO: 3 - protein, 203 aa, Unknown
  methanotrophs
VRVHPKWNETMKVVSNFLEVGEYNAIAATGMLWDSAQAAEQKNGYLAQVLDEIRHTHQCAYVNYYFAKNGQDPAGHNDARRTRTIGPLWKGMKRVFSDGFISGDAVECSLNLQLVGEACFTNPLIVAVTEWAAANGDEITPTVFLSIETDELRHMANGYQTVVSIANDPASAKYLNTDLNNAFWTQQKYFTPVLGMLFEYGSK

SEQ ID NO: 4 - protein, 140 aa, Nitrosomonas europaea
HGERSQEPFLRMRTVQWYDIKWGPEVTKVNENAKITGKFHLAEDWPRAAAQPDFSFFNVGSPSPVFVRLSTKINGHPWFISGPLQIGRDYEFEVNLRARIPGRHHMHAMLNVKDAGPIAGPGAWMNITGSWDDFTNPLKL

SEQ ID NO: 5 - protein, 250 aa, Methylococcus capsulatus
CRISFGEVGSFEAEVVGLNWVSSNTVQFLLQKRPDECGNRGVKFEPGQFMDLTIPGTDVSRSYSPANLPNPEGRLEFLIRVLPEGRFSDYLRNDARVGQVLSVKGPLGVFGLKERGMAPRYFVAGGTGLAPVVSMVRQMQEWTAPNETRIYFGVNTEPELFYIDELKSLERSMRNLTVKACVWHPSGDWEGEQGSPIDALREDLESSDANPDIYLCGPPGMIDAACELVRSRGIPGEQVFFEKFLPSGAA

SEQ ID NO: 6 - protein, 348 aa, Methylococcus capsulatus
MQRVHTITAVTEDGESLRFECRSDEDVITAALRQNIFLMSSCREGGCATCKALCSEGDYDLKGCSVQALPPEEEEEGLVLLCRTYPKTDLEIELPYTHCRISFGEVGSFEAEVVGLNWVSSNTVQFLLQKRPDECGNRGVKFEPGQFMDLTIPGTDVSRSYSPANLPNPEGRLEFLIRVLPEGRFSDYLRNDARVGQVLSVKGPLGVFGLKERGMAPRYFVAGGTGLAPVVSMVRQMQEWTAPNETRIYFGVNTEPELFYIDELKSLERSMRNLTVKACVWHPSGDWEGEQGSPIDALREDLESSDANPDIYLCGPPGMIDAACELVRSRGIPGEQVFFEKFLPSGAA

SEQ ID NO: 7 - protein, 140 aa, Unknown
  methanotrophs
SVNSNAYDAGIMGLKGKDFADQFFADENQVVHESDTVVLVLKKSDEINTFIEEILLTDYKKNVNPTVNVEDRAGYWWIKANGKIEVDCDEISELLGRQFNVYDFLVDVSSTIGRAYTLGNKFTITSELMGLDRKLEDYHA

SEQ ID NO: 8 - protein, 140 aa, Unknown
  methanotrophs
AHYDELKRDLGMLESTITFKNGLTYARGITSSVDVLFDYVNFQRGLLESIEDCDVEIKGNAKIWWYGARDEVNVTPNVNKKYDTLLIEEIFTNIEDSKKLVLVVTDSEHVVQNEDAFFQDAFDKGKLGMIGADYANSNVS